(19) United States
(12) Patent Application Publication
(10) Pub. No.: US 2023/0035466 A1
(43) Pub. Date: Feb. 2, 2023

(54) SYSTEM AND METHOD FOR SPRAY TAN CUSTOMIZATION USING MULTIPART TANNING SOLUTION

(71) Applicant: SUNLESS, INC., Macedonia, OH (US)

(72) Inventors: Benjamin David Johnson, Northfield, OH (US); Cari Jo Gerbracht, Kirtland, OH (US); Douglas Alan Goeke, Bethesda, MD (US)

(73) Assignee: SUNLESS, INC., Macedonia, OH (US)

(21) Appl. No.: 17/873,612

(22) Filed: Jul. 26, 2022

Related U.S. Application Data
(60) Provisional application No. 63/226,483, filed on Jul. 28, 2021.

(51) Int. Cl. B05B 7/26; A61M 35/00; B05B 12/14; B01F 33/84; B05B 16/20

(52) U.S. Cl. CPC B05B 7/26; A61M 35/25; B05B 12/1418; B01F 33/8442; B05B 16/20; B01F 2101/21

(57) ABSTRACT

A system and method for customizing the solution used in the sunless spray tanning process are provided. In at least one form, the solution is volumetrically mixed at the point of use, allowing for greater customization and a more consistent tanning result. Providing improved customization at the point of use is accomplished, in at least one form, by using a two-part tanning solution: one part being a high concentration DHA solution and another part being an activator solution. The two parts are mixed with one another at the point of use based on user preferences at the time of the tanning session to provide a specific final DHA concentration for a customized tanning result.

The present application is based on and claims priority to U.S. Provisional Application Ser. No. 63/226,483, filed Jul. 28, 2021, which is incorporated herein by reference in its entirety.

### TECHNICAL FIELD

The present exemplary embodiments relate to the spray tan field. They find particular application in conjunction with spray tan products and techniques for spray tan application and will be described with particular reference thereto. However, it is to be appreciated that the present exemplary embodiment is also amenable to other like applications.

### BACKGROUND

Sunless spray tanning typically utilizes an aqueous solution of dihydroxyacetone (DHA) to temporarily darken surface layer skin cells and simulate a tan. Currently, DHA solutions are manufactured with a set concentration of DHA that determines the darkness of the tan once the solution is applied on the skin of a user or tanner. Typical spray tanning solutions manufactured with such a fixed concentration of DHA result in providing the tanner with only 2 or 3 choices for the darkness of their tan. This group of choices may not give the precise look desired by the tanner, especially given the variation in skin tone from person to person. In addition, less popular concentrations that provide very light or very dark results may not be consumed quickly enough to avoid product spoilage.

The current accepted approach in the industry is to provide various concentrations of DHA solution that can be used in a specific type of spray tan booth. Different products may include different ingredients for appearance, scent, skin care or other desirable traits. These ingredients are, however, all incorporated into the formulation during the manufacturing process.

Some tanning methods allow additional ingredients, typically referred to as “add-ons”, to be mixed at the time of purchase. However, add-on ingredients are typically limited to scents, bronzers, and/or accelerators that require low volume and alone do not affect the final tanning result. The add-on ingredients plus the liquids carrying them often have a volume of, e.g., 1 to 5 ml. Scents provide a pleasant fragrance during and after the tan. Bronzers are pigments that provide immediate color during the spray session. Accelerators are additives that speed the tanning process.

### BRIEF DESCRIPTION

In one aspect of the presently described embodiments, a system for spray tan customization using a multi-part solution comprises a first reservoir configured to stage a first solution comprising dihydroxyacetone (DHA), the first reservoir being provided with a first pump, a second reservoir configured to stage a second solution comprising activating components including amino acids, the second reservoir being provided with a second pump, a third reservoir configured to receive the first and second solution, and a controller including at least one processor and at least one memory, wherein the at least one memory stores code or instructions that can be executed by the at least one processor to initiate a mix sequence for the first solution staged in the first reservoir and the second solution staged in the second reservoir based on user preferences for a spray tan session, activate the first pump to dispense the first solution from the first reservoir to a third reservoir based on the user preferences and activate the second pump to dispense the second solution from the second reservoir to the third reservoir based on the user preferences wherein the first solution and the second solution are mixed in the third reservoir.

In another aspect of the presently described embodiments, the system further comprises a mixing element configured to mix contents of the third reservoir.

In another aspect of the presently described embodiments, the controller is further configured to activate the mixer to mix the first solution and the second solution in the third reservoir.

In another aspect of the presently described embodiments, the system further comprises a fourth reservoir to collect additional ingredients, the fourth reservoir being provided with a third pump.

In another aspect of the presently described embodiments, the system further comprises a check valve between the third pump and the third reservoir.

In another aspect of the presently described embodiments, the system further comprises a solenoid switch connected to the third reservoir, the solenoid switch being configured to trigger water flow into the third reservoir.

In another aspect of the presently described embodiments, the second pump is activated to dispense the second solution to the third reservoir through the fourth reservoir.

In another aspect of the presently described embodiments, the third pump is activated by the controller to dispense contents of the fourth reservoir to the third reservoir based on the user preferences.

In another aspect of the presently described embodiments, the system further comprises a fifth reservoir including moisturizer, the fifth reservoir being provided with a fourth pump.

In another aspect of the presently described embodiments, the fourth pump is selectively activated by the controller to dispense contents of the fifth reservoir to the third reservoir.

In another aspect of the presently described embodiments, mixed contents of the third reservoir are provided to a spray tan booth.

In another aspect of the presently described embodiments, mixed contents of the third reservoir are provided to a spray gun.

In another aspect of the presently described embodiments, a method for spray tan customization using multi-part solution comprises initiating a mix sequence for a first solution comprising dihydroxyacetone (DHA) staged in a first reservoir and a second solution comprising activating components including amino acids staged in a second reservoir based on user preferences for a spray tan session, dispensing the first solution from the first reservoir to a third reservoir based on the user preferences, dispensing the second solution from the second reservoir to the third reservoir based on the user preferences and mixing the first solution and the second solution in the third reservoir.

In another aspect of the presently described embodiments, the mixing comprises activating a mixer to mix the first solution and the second solution in the third reservoir.

In another aspect of the presently described embodiments, the method further comprises collecting additional ingredients in a fourth reservoir.

In another aspect of the presently described embodiments, the dispensing the second solution from the second reservoir to the third reservoir comprises dispensing the second solution through the fourth reservoir to the third reservoir.

In another aspect of the presently described embodiments, the method further comprises dispensing contents from the fourth reservoir to the third reservoir based on the user preferences.

In another aspect of the presently described embodiments, the method further comprises dispensing moisturizer.

In another aspect of the presently described embodiments, the method further comprises providing mixed contents of the third reservoir to a spray tan booth.

In another aspect of the presently described embodiments, the method further comprises providing contents of the third reservoir to a spray gun.

### BRIEF DESCRIPTION OF THE DRAWINGS

FIG. 1(a) is a representative illustration of an example system according to the presently described embodiments;

FIG. 1(b) is a representative illustration of an example system according to the presently described embodiments;

FIG. 1(c) is a representative illustration of an example system according to the presently described embodiments;

FIG. 1(d) is a representative illustration of an example system according to the presently described embodiments;

FIG. 1(e) is a representative illustration of an example system according to the presently described embodiments;

FIG. 1(f) is a representative illustration of an example system according to the presently described embodiments;

FIG. 2 is a flow chart illustrating an example method according to the presently described embodiments; and,

FIG. 3 is a flow chart illustrating an example method according to the presently described embodiments.

### DETAILED DESCRIPTION

According to the presently described embodiments, a new approach to customizing the solution used in the sunless spray tanning process is provided. In at least one form of the presently described embodiments, the solution is volumetrically mixed at the point of use, allowing for greater customization and a more consistent tanning result.

The provision of such greater customization at the point of use is accomplished, in at least one form, by using a two-part tanning solution: one part (e.g., sometimes also referred to herein as Part A or Part A solution) being a high concentration DHA solution and another part (e.g., sometimes also referred to herein as Part B or Part B solution) being an “activator” to activate the DHA, when mixed. This Part B solution, in at least one form, also includes an “optimizer” to help create an even tone on skin when applied. According to the presently described embodiments, the two parts, Part A and Part B, are mixed with one another at the point of use based on user preferences at the time of the tanning session to provide a specific final DHA concentration for a customized tanning result.

As noted above, currently, solution characteristics are set by the manufacturer at the time of production. Because the reaction that allows DHA to be used as the tanning agent takes time (i.e., approximately twenty (20) minutes), on-demand mixing was typically discounted as a possible approach. Also, due to the chemical nature of DHA, mixing at the point of use has not heretofore seemed feasible.

Furthermore, the exact concentration of ingredients is important to the tanning result. In addition, depending on location, there may be restrictions on the DHA concentration in a solution. For example, the United States and the European Union have different regulations about total allowable concentration. These values can also change over time as laws and regulations change. This gives the solution manufacturer incentive to control the exact concentration of ingredients within their production processes.

Further, solutions controlled during production have been sufficient to offer three or four different tan levels and, in the past, this has been enough for the users. It would, however, be desirable for the users to have the ability to achieve a higher number of tan levels at the point of use.

As such, according to the presently described embodiments, a premixed DHA solution (e.g., Part A) is provided for use in the system (e.g., a booth or hand-held device). The premixing may be achieved, for example, at a manufacturing or provider facility using any of a variety of suitable techniques and subsequently provided to the point of use. In one example, water and DHA powder (often stored at cooler temperatures) are mixed to obtain a solution having a desired DHA concentration, packaged, and shipped to the point of use. Of course, while the Part A solution is, in the presently described embodiments, a high concentration DHA containing liquid, those skilled in the art will appreciate that other ingredients may also be included in the Part A solution by the provider or manufacturer to achieve different effects depending on the features and/or characteristics of the desired final product. For example, the Part A solution may also include: crotonaldehyde, pyruvaldehyde, glycolaldehyde, glutaraldehyde, otho-phthaldehyde, sorbose, fructose, erythrulose, methylvinylketone, food coloring, or any other available colorant, as described in e.g., US Patent Publication No. 201010122745A1, filed Nov. 23, 2009 as U.S. application Ser. No. 12/624,083, which is hereby incorporated by reference.

The Part B solution, in at least one form, is also premixed at the manufacturer or provider facility and then provided to the system at the point of use. The Part B solution may take a variety of forms but, in at least one form, will be formulated to be mixed, at the point of use, with the Part A solution according to user selection of a tan level. In at least one form according to the presently described embodiments, the Part B solution contains no DHA. However, amino acids are included in the Part B solution to activate the DHA/skin tanning reaction once mixed with the Part A solution. The Part A solution contains no activating amino acids. Also, any suitable amino acid that activates the DHA can be used as an activating component. Further, in Part B solutions including optimizer components, any suitable optimizer may be used.

Keeping the Part A and Part B solutions separate from one another until the point of use has many benefits in the context of the presently described embodiments, including those described herein. It also has the ancillary benefit of extending the life of the DHA solution. In this regard, because the presence of amino acids activates the DHA reaction, once mixed, DHA will slowly decay over time, even in solution. Still further, another consequence of keeping the ingredients separate is that there is a reduction in odor as part of the tanning process. In this regard, the DHA tanning reaction with skin has been known to generate a slight odor. Some users dislike this odor. Typically, the manufacturer will use techniques or additives to counteract the odor. However, mixing the Part A and Part B solutions at the point of use, in accord with the presently described embodiments, results in a reduction of this odor generation without the additional techniques or additives.

Although as noted, the Part A solution contains no activating amino acids and the Part B solution contains no DHA, some ingredients may be included in both parts, or either part, at the option of the manufacturer. For example, moisturizing agents, such as hyaluronic acid, sodium PCA (pyrrolidone carboxylic acid), and glycerin, may be included in one or both of the Part A and Part B solutions.

With this system according to the presently described embodiments, users will be able to achieve a higher number of tan levels at the point of use than could be previously provided. In this regard, based on the on-demand selection of a tan level or shade at the point of use by the user, an appropriate amount of Part A and Part B solutions are mixed by the system.

Furthermore, the detrimental effects of adding volume to the mix of Part A and Part B solutions from optional add-ons, particularly at the point of use, has not been well understood with regards to tan quality. As noted above, add-ons alone do not impact the final tan result. However, add-ons are typically premixed with other material to form solutions and then these add-on solutions are provided to the system at the point of use. This extra volume of add-on solution may well impact the final desired tan result. For example, the solutions into which add-ons are mixed may or may not be included in the volumetric load and, thus, create uncertainty in planning, e.g., planning to maintain DHA concentration at desired levels for consistent tan levels. As will be appreciated by those skilled in the art, if a typical tanning session uses a final solution having a volume of 125 ml, an add-on solution having, for example, a volume of 5 ml, 10 ml, 15 ml or 20 ml, is not insignificant—and could impact the final tan result sufficiently to create user dissatisfaction.

To address this problem according to the presently described embodiments, the amounts of Part A and/or Part B solutions may be adjusted so that the desired concentration of DHA is maintained and remains predictable. In one example, the volume of Part B solution may be reduced by the volume of add-on solution to maintain the desired concentration of DHA (or Part A) solution. In another example, the volume of Part A solution may be increased based on the volume of add-on solution to maintain the DHA concentration. This alternative will increase the volume of total solution so it could then be applied over a longer period of time.

Thus, paying closer attention to the current process, including the precise DHA concentration levels, sheds light on these issues and the presently described embodiments provide an elegant solution not possible with existing technologies.

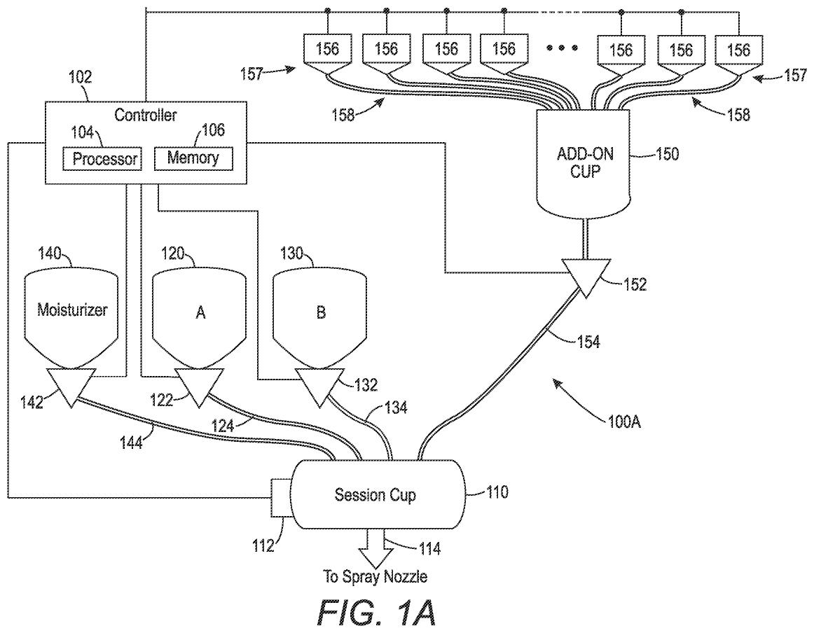

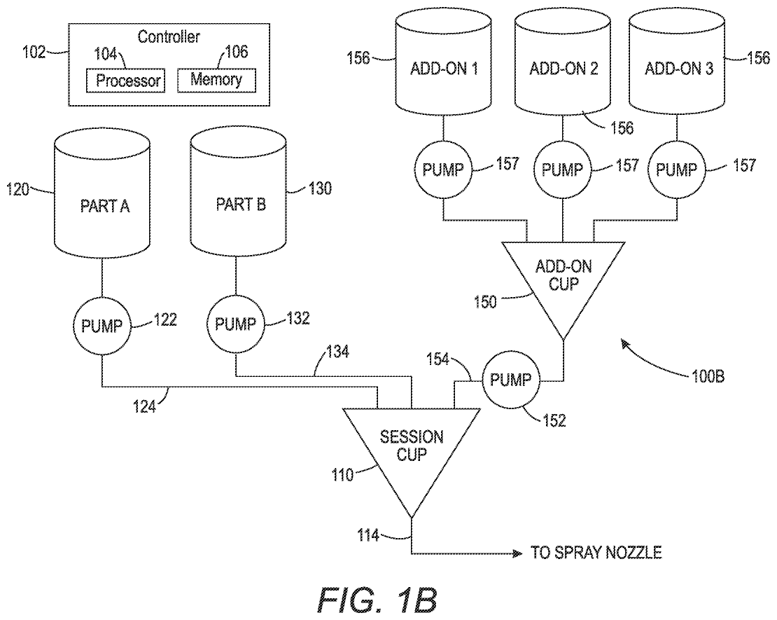

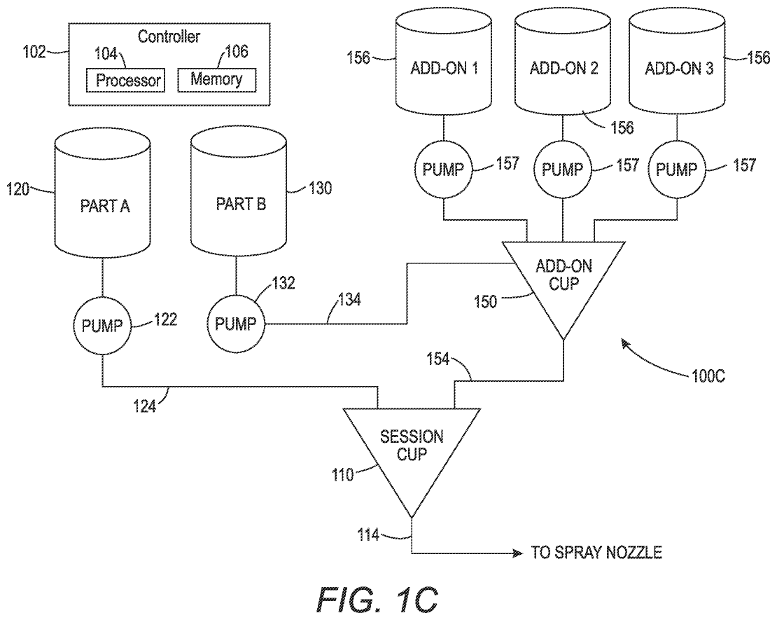

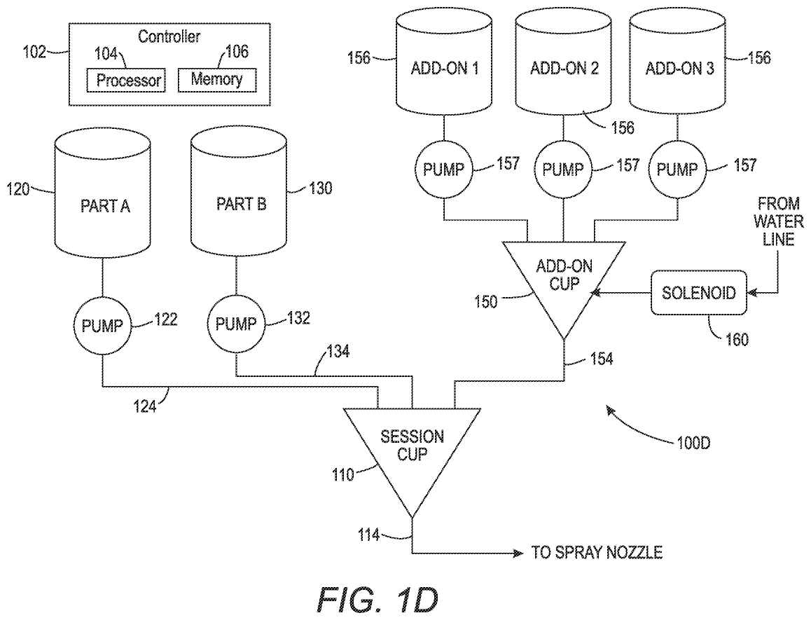

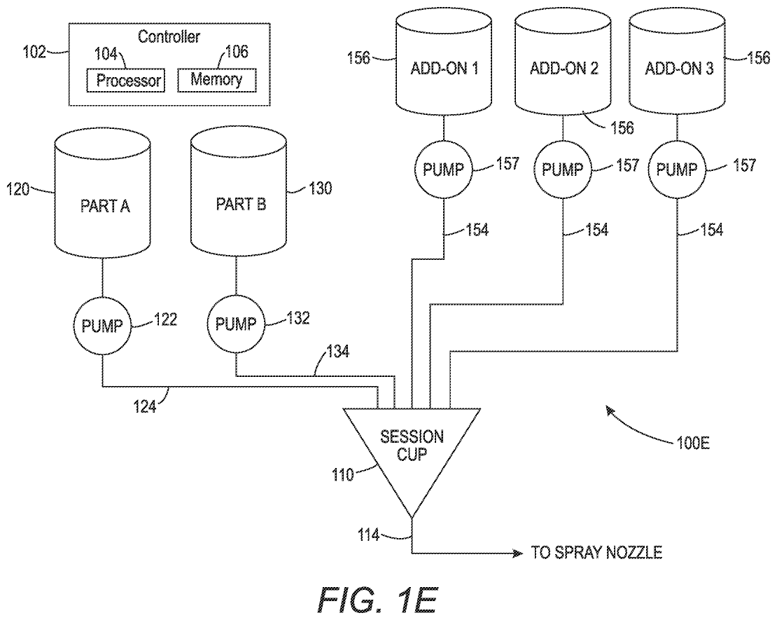

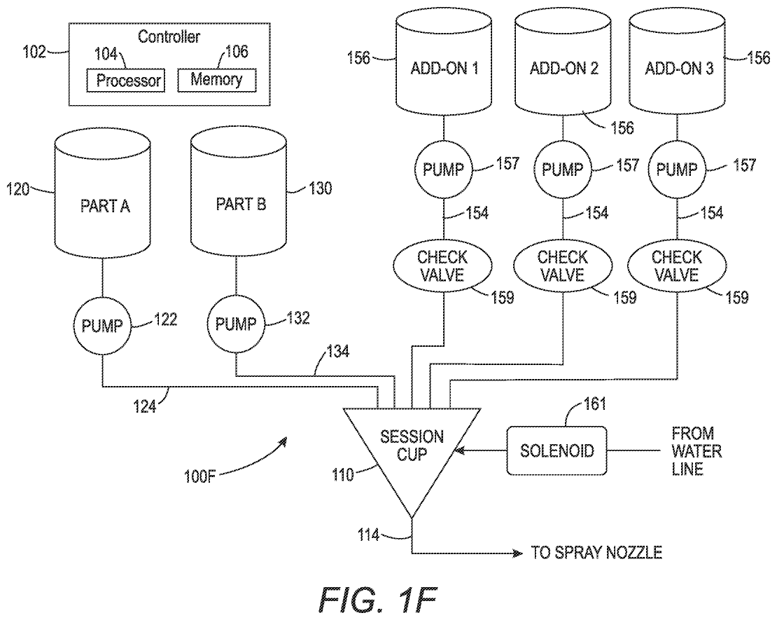

With reference now to FIG. 1(a), an example system 100A for spray tan customization using a multi-part solution is illustrated. As shown, the system 100A includes a controller 102 having at least one processor 104 and at least one memory 106. Of course, the at least one processor 104 is configured to execute code, instructions or routines that may be stored on the at least one memory 106 (or on other appropriate memories) to trigger or cause components of the system 100A (including, but not limited to, the controller 102, processor 104 and/or memory 106 or other elements) to perform or function in suitable manners to implement the presently described embodiments and other objectives.

It will be appreciated by those of skill in the art, upon a reading of the present specification, that the controller 102 as described, as well as the described processors and memories, may take a variety of forms to implement the presently described embodiments. The processors can be embodied in a variety of hardware forms, such as digital processors, single-core processors, multi-core processors, or coprocessors, or the like. The memories may be any type of tangible non-transitory computer readable medium such as random-access memory (RAM), read only memory (ROM), magnetic disk or tape, optical disk, flash memory, or holographic memory, or the like. In at least one embodiment, the memory 106 may comprise a combination of random-access memory and read only memory portions.

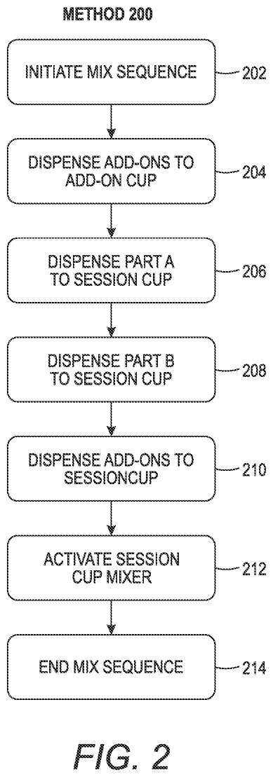

Also shown in FIG. 1(a) is a session cup or reservoir 110 and associated mixing element or mixer 112. The session cup 110 is configured and positioned to receive suitable amounts of solution and/or other fluids (such as moisturizers and add-ons) for a tanning session. According to the presently described embodiments, the tanning session is customized according to the preferences of the user or tanner as described above, for example. As such, the precise amounts of solution and/or other fluids will be controlled by the system (e.g., by way of the controller 102) and likely vary from session to session, again, as described above, for example. In at least one form, once suitable amounts of solution are provided to the session cup 110, as will be described in detail in connection with FIG. 2, the mixing element or mixer 112 is activated to mix or provide turbulence to the ingredients to achieve a solution, if necessary. In the cases where a mixer or mixing element is used, the mixer or mixing element may take a variety of forms including, for example, a physical agitator, a vibrational agitator or an acoustic agitator. As will be described in connection with other embodiments, however, the mixing element or mixer 112 is optional or may not even be necessary where the technique of providing the solutions to the session cup 110 has sufficient turbulence to sufficiently mix the solutions. In either case, the solution is then dispensed appropriate through outlet 114 to, for example, an appropriate spray nozzle.

As noted, solution and other fluids are provided to the session cup 110. For example, a first solution or Part A is provided to the session cup 110 from a supply or reservoir 120 by a pump or flow control device or valve 122 through fluid line 124. It will be appreciated that, in at least one form of the presently described embodiments, the Part A solution is a high concentration DHA solution. A second solution or Part B is provided to the session cup 110 from a supply or reservoir 130 by a pump or flow control device or valve 132 through fluid line 134. It will be appreciated that, in at least one form of the presently described embodiments, the Part B solution is a solution having activating and optimizing components.

The supply or reservoir 120 and the supply of reservoir 130 may take a variety of forms. Generally, the supply or reservoir 120 and the supply or reservoir 130 are configured to stage or store the first solution (or Part A solution) and the second solution (or Part B solution), respectively. In one further example, these elements are configured as one-time use items and each take the form of a bag-in-a-box. Other disposable configurations may also be used. In some forms, these disposable-type systems also utilize a secure solution load and/or unload sequence to ensure proper installation of the correct solution. Various forms of security measures, including Radio Frequency Identification (RFID) tags, bar codes and/or suitable locks, may be used. Alternatively, a refillable arrangement may be utilized which may or may not utilize a secure sequence for the refilling process.

It will be further appreciated that a moisturizer is provided, at appropriate times and typically alone (as will be described hereafter), to the session cup 110 from a supply or reservoir 140 by a pump or flow control device or valve 142 through fluid line 144.

Add-on ingredients, or add-ons, in some circumstances, may also be added to the session cup 110 at appropriate times. In this regard, add-ons are provided to the session cup 110 from add-on cup or reservoir 150 by a pump or fluid control device or valve 152 through fluid line 154. The add-on cup or reservoir 150 is selectively supplied with, and is configured to collect, add-on ingredients such as bronzers, accelerators, and scents dispensed from individual supply elements 156 of those respective ingredients through supply lines 158 using corresponding pumps 157. As shown, the number of add-on supply elements 156 and supply lines 158, and the supply of the corresponding ingredients, may vary from system to system. In at least one form of the presently described embodiments, the add-on cup 150 has a volume of approximately 20 ml and the ad-on supply elements 156 each provide a volume of 1 ml to 5 ml of its add-on ingredient to the add-on cup 150.

As with the supply or reservoirs 120 and 130, the supply or reservoir 140, add-on cup 150 and add-on supply elements 156 may take a variety of forms. They could be disposable in nature or refillable. Also, in some forms, a secure installation or refilling sequence may be implemented for these items.

Also shown in FIG. 1(a) are connections between various components. While representatively shown as continuous lines in FIG. 1(a), the actual connections or network may take a variety of different forms to suit the implementation or objectives of the system design. For example, hardwire connections may be implemented. Alternatively, or in combination, wireless connections may be established.

In operation, according to at least one form of the presently described embodiments, a liquid, high concentration DHA solution (Part A) of supply or reservoir 120 is used as the base tanning solution. A second solution with activating and optimizing components (Part B) from supply or reservoir 130 is used to dilute the high concentration DHA solution to provide the precise final concentration required for the desired tanning results. Generally, it should be appreciated that higher concentrations of DHA yield darker tans. The volume of Part B solution supplied from supply or reservoir 130 can be further modified to account for the presence of add-ons from add-on cup 150 for fragrances, bronzers, accelerators or other solutions that provide skincare benefits or a faster tan. In this regard, by using less of the Part B solution to account for the liquid volume added by the optional add-ons, the concentration of DHA in the final solution mixture in the session cup 110 remains constant, yielding more consistent tanning results.

Dosing pumps, such as pumps 122, 132, and 152, as well as dosing pumps 142 and 157, provide a predetermined amount of solution based on the settings for the session. In at least one form, the pumps are highly accurate or high precision pumps to provide very precisely measured amounts. Such precision is advantageous as described in connection with the presently described embodiments where maintaining the DHA concentration is desired. Each solution (e.g., Part A, Part B, and add-ons) is dispensed independently by its own pump, but with the final total volume fixed by an electromechanical logic program. This program is, for example, stored and executed by the controller 102, as alluded to above. Among other functions, this program or routine handles the math required to balance the solutions, allowing the user to simply choose a tan level, with gradations down to 1 ml.

With reference now to FIG. 1(b), another example system, system 100B, for spray tan customization using a multi-part solution is illustrated. As shown, the system 100B includes a controller 102 having at least one processor 104 and at least one memory 106, as described in connection with FIG. 1(a).

Also shown in FIG. 1(b) is a session cup or reservoir 110. The session cup 110 is configured and positioned to receive suitable amounts of solution and/or other fluids (such as moisturizers and add-ons) for a tanning session. According to the presently described embodiments, the tanning session is customized according to the preferences of the user or tanner. As such, the precise amounts of solution and/or other fluids will be controlled by the system (e.g., by way of the controller 102) and likely vary from session to session. In at least one form, the provision of the solutions to the session cup 110 has sufficient turbulence to sufficiently mix the solutions. That is, a mixing element or mixer is not provided. The solution is then dispensed appropriate through outlet 114.

As noted, solution and other fluids are provided to the session cup 110. For example, a first solution or Part A is provided to the session cup 110 from a supply or reservoir 120 by a pump or flow control device or valve 122 through fluid line 124. It will be appreciated that, in at least one form of the presently described embodiments, the Part A solution is a high concentration DHA solution. A second solution or Part B is provided to the session cup 110 from a supply or reservoir 130 by a pump or flow control device or valve 132 through fluid line 134. It will be appreciated that, in at least one form of the presently described embodiments, the Part B solution is a solution having activating and optimizing components.

The supply or reservoir 120 and the supply of reservoir 130 may take a variety of forms, as described in more detail in connection with FIG. 1(a). However, generally, the supply or reservoir 120 and the supply or reservoir 130 are configured to stage or store the first solution (or Part A solution) and the second solution (or Part B solution), respectively.

It will be further appreciated that, although not shown in FIG. 1(b), a moisturizer may be provided, in at least some forms, at appropriate times and typically alone (as will be described hereafter), to the session cup 110 from a supply or reservoir by a pump or flow control device or valve through fluid line, as described, for example in FIG. 1(a).

Add-on ingredients, or add-ons, in some circumstances, may also be added to the session cup 110 at appropriate times. In this regard, add-ons are provided to the session cup 110 from add-on cup or reservoir 150 by a pump or fluid control device or valve 152 through fluid line 154, as described in connection with FIG. 1(a). The add-ons are provided to the add-on cup 150 from supply elements or reservoirs 156 via corresponding pumps 157, as described in connection with FIG. 1(a).

Electrical connections between various components are not shown in FIG. 1(b) for ease of reference. As an example, the lines of FIG. 1(a) may be implemented. However, it should be appreciated that the actual connections or network may take a variety of different forms to suit the implementation or objectives of the system design. For example, hardwire connections may be implemented. Alternatively, or in combination, wireless connections may be established.

In operation, according to at least one form of the presently described embodiments, a liquid, high concentration DHA solution (Part A) of supply or reservoir 120 is used as the base tanning solution. A second solution with activating and optimizing components (Part B) from supply or reservoir 130 is used to dilute the high concentration DHA solution to provide the precise final concentration required for the desired tanning results. Generally, it should be appreciated that higher concentrations of DHA yield darker tans. The volume of Part B solution supplied from supply or reservoir 130 can be further modified to account for the presence of add-ons from add-on cup 150 for fragrances, bronzers, accelerators or other solutions that provide skincare benefits or a faster tan. In this regard, by using less of the Part B solution to account for the liquid volume added by the optional add-ons, the concentration of DHA in the final solution mixture in the session cup 110 remains constant, yielding more consistent tanning results.

Dosing pumps, such as pumps 122, 132, and 152, as well as dosing pumps 157, provide a predetermined amount of solution based on the settings for the session and are described in greater detail in connection with FIG. 1(a).

With reference now to FIG. 1(c), another example system, system 100C, for spray tan customization using a multi-part solution is illustrated. As shown, the system 100C includes a controller 102 having at least one processor 104 and at least one memory 106, as described in connection with FIG. 1(a).

Also shown in FIG. 1(c) is a session cup or reservoir 110. The session cup 110 is configured and positioned to receive suitable amounts of solution and/or other fluids (such as moisturizers and add-ons) for a tanning session. According to the presently described embodiments, the tanning session is customized according to the preferences of the user or tanner. As such, the precise amounts of solution and/or other fluids will be controlled by the system (e.g., by way of the controller 102) and likely vary from session to session. In at least one form, the provision of the solutions to the session cup 110 has sufficient turbulence to sufficiently mix the solutions. That is, a mixing element or mixer is not provided. The solution is then dispensed appropriate through outlet 114.

As noted, solution and other fluids are provided to the session cup 110. For example, a first solution or Part A is provided to the session cup 110 from a supply or reservoir 120 by a pump or flow control device or valve 122 through fluid line 124. It will be appreciated that, in at least one form of the presently described embodiments, the Part A solution is a high concentration DHA solution. A second solution or Part B is provided from a supply or reservoir 130 by a pump or flow control device or valve 132 through fluid line 134. However, in this embodiment, the second solution, or Part B solution, is routed into an add-on cup 150 before being provided to the session cup 110. This configuration allows the second solution to be used as a purge solution to purge heavy bronzers and other residue from the add-on cup 150 and corresponding delivery lines at appropriate times. It will be appreciated that, in at least one form of the presently described embodiments, the Part B solution is a solution having activating and optimizing components.

The supply or reservoir 120 and the supply of reservoir 130 may take a variety of forms, as described in more detail in connection with FIG. 1(a). However, generally, the supply or reservoir 120 and the supply or reservoir 130 are configured to stage or store the first solution (or Part A solution) and the second solution (or Part B solution), respectively.

It will be further appreciated that, although not shown in FIG. 1(c), a moisturizer may be provided, in at least some forms, at appropriate times and typically alone (as will be described hereafter), to the session cup 110 from a supply or reservoir by a pump or flow control device or valve through fluid line, as described, for example in FIG. 1(a).

Add-on ingredients, or add-ons, in some circumstances, may also be added to the session cup 110 at appropriate times. In this regard, add-ons are provided to the session cup 110 from add-on cup or reservoir 150 through fluid line 154, as described in connection with FIG. 1(a). The add-ons are provided to the add-on cup 150 from supply elements or reservoirs 156 via corresponding pumps 157, as described in connection with FIG. 1(a). In this embodiment, the add-ons may be mixed with the second solution as they are delivered to the session cup 110.

Electrical connections between various components are not shown in FIG. 1(b) for ease of reference. As an example, the lines of FIG. 1(a) may be implemented. However, it should be appreciated that the actual connections or network may take a variety of different forms to suit the implementation or objectives of the system design. For example, hardwire connections may be implemented. Alternatively, or in combination, wireless connections may be established.

In operation, according to at least one form of the presently described embodiments, a liquid, high concentration DHA solution (Part A) of supply or reservoir 120 is used as the base tanning solution. A second solution with activating and optimizing components (Part B) from supply or reservoir 130 is used to dilute the high concentration DHA solution to provide the precise final concentration required for the desired tanning results. Generally, it should be appreciated that higher concentrations of DHA yield darker tans. The volume of Part B solution supplied from supply or reservoir 130 can be further modified to account for the presence of add-ons from add-on cup 150 for fragrances, bronzers, accelerators or other solutions that provide skincare benefits or a faster tan. In this regard, by using less of the Part B solution to account for the liquid volume added by the optional add-ons, the concentration of DHA in the final solution mixture in the session cup 110 remains constant, yielding more consistent tanning results.

Dosing pumps, such as pumps 122, 132, and 157 provide a predetermined amount of solution based on the settings for the session and are described in greater detail in connection with FIG. 1(a).

With reference now to FIG. 1(d), another example system, system 100D, for spray tan customization using a multi-part solution is illustrated. As shown, the system 100D includes a controller 102 having at least one processor 104 and at least one memory 106, as described in connection with FIG. 1(a).

Also shown in FIG. 1(d) is a session cup or reservoir 110. The session cup 110 is configured and positioned to receive suitable amounts of solution and/or other fluids (such as moisturizers and add-ons) for a tanning session. According to the presently described embodiments, the tanning session is customized according to the preferences of the user or tanner. As such, the precise amounts of solution and/or other fluids will be controlled by the system (e.g., by way of the controller 102) and likely vary from session to session. In at least one form, the provision of the solutions to the session cup 110 has sufficient turbulence to sufficiently mix the solutions. That is, a mixing element or mixer is not provided. The solution is then dispensed appropriate through outlet 114.

As noted, solution and other fluids are provided to the session cup 110. For example, a first solution or Part A is provided to the session cup 110 from a supply or reservoir 120 by a pump or flow control device or valve 122 through fluid line 124. It will be appreciated that, in at least one form of the presently described embodiments, the Part A solution is a high concentration DHA solution. A second solution or Part B is provided to the session cup 110 from a supply or reservoir 130 by a pump or flow control device or valve 132 through fluid line 134. It will be appreciated that, in at least one form of the presently described embodiments, the Part B solution is a solution having activating and optimizing components.

The supply or reservoir 120 and the supply of reservoir 130 may take a variety of forms, as described in more detail in connection with FIG. 1(a). However, generally, the supply or reservoir 120 and the supply or reservoir 130 are configured to stage or store the first solution (or Part A solution) and the second solution (or Part B solution), respectively.

It will be further appreciated that, although not shown in FIG. 1(d), a moisturizer may be provided, in at least some forms, at appropriate times and typically alone (as will be described hereafter), to the session cup 110 from a supply or reservoir by a pump or flow control device or valve through fluid line, as described, for example in FIG. 1(a).

Add-on ingredients, or add-ons, in some circumstances, may also be added to the session cup 110 at appropriate times. In this regard, add-ons are provided to the session cup 110 from add-on cup or reservoir 150 through fluid line 154, as described in connection with FIG. 1(a). The add-ons are provided to the add-on cup 150 from supply elements or reservoirs 156 via corresponding pumps 157, as described in connection with FIG. 1(a). In this embodiment, there is no pump provided to the add-on cup 150; however, a solenoid switch 160 (or another suitable switch) is provided to allow water flow, at appropriate times, through the add-on cup 150 to suitably purge the system. This could be controlled in a variety of ways including, for example, by the control system or controller 102. It should also be appreciated that the solenoid switch (or another suitable switch) controlling water flow for purging, for example, the add-on cup could be implemented in other embodiments employing an add-on cup (e.g., FIG. 1(a), FIG. 1(b), and FIG. 1(c)).

Electrical connections between various components are not shown in FIG. 1(d) for ease of reference. As an example, the lines of FIG. 1(a) may be implemented. However, it should be appreciated that the actual connections or network may take a variety of different forms to suit the implementation or objectives of the system design. For example, hardwire connections may be implemented. Alternatively, or in combination, wireless connections may be established.

In operation, according to at least one form of the presently described embodiments, a liquid, high concentration DHA solution (Part A) of supply or reservoir 120 is used as the base tanning solution. A second solution with activating and optimizing components (Part B) from supply or reservoir 130 is used to dilute the high concentration DHA solution to provide the precise final concentration required for the desired tanning results. Generally, it should be appreciated that higher concentrations of DHA yield darker tans. The volume of Part B solution supplied from supply or reservoir 130 can be further modified to account for the presence of add-ons from add-on cup 150 for fragrances, bronzers, accelerators or other solutions that provide skincare benefits or a faster tan. In this regard, by using less of the Part B solution to account for the liquid volume added by the optional add-ons, the concentration of DHA in the final solution mixture in the session cup 110 remains constant, yielding more consistent tanning results.

Dosing pumps, such as pumps 122, 132, and 157 provide a predetermined amount of solution based on the settings for the session and are described in greater detail in connection with FIG. 1(a).

With reference now to FIG. 1(e), another example system, system 100E, for spray tan customization using a multi-part solution is illustrated. As shown, the system 100E includes a controller 102 having at least one processor 104 and at least one memory 106, as described in connection with FIG. 1(a).

Also shown in FIG. 1(e) is a session cup or reservoir 110. The session cup 110 is configured and positioned to receive suitable amounts of solution and/or other fluids (such as moisturizers and add-ons) for a tanning session. According to the presently described embodiments, the tanning session is customized according to the preferences of the user or tanner. As such, the precise amounts of solution and/or other fluids will be controlled by the system (e.g., by way of the controller 102) and likely vary from session to session. In at least one form, the provision of the solutions to the session cup 110 has sufficient turbulence to sufficiently mix the solutions. That is, a mixing element or mixer is not provided. The solution is then dispensed appropriate through outlet 114.

As noted, solution and other fluids are provided to the session cup 110. For example, a first solution or Part A is provided to the session cup 110 from a supply or reservoir 120 by a pump or flow control device or valve 122 through fluid line 124. It will be appreciated that, in at least one form of the presently described embodiments, the Part A solution is a high concentration DHA solution. A second solution or Part B is provided to the session cup 110 from a supply or reservoir 130 by a pump or flow control device or valve 132 through fluid line 134. It will be appreciated that, in at least one form of the presently described embodiments, the Part B solution is a solution having activating and optimizing components.

The supply or reservoir 120 and the supply of reservoir 130 may take a variety of forms, as described in more detail in connection with FIG. 1(a). However, generally, the supply or reservoir 120 and the supply or reservoir 130 are configured to stage or store the first solution (or Part A solution) and the second solution (or Part B solution), respectively.

It will be further appreciated that, although not shown in FIG. 1(e), a moisturizer may be provided, in at least some forms, at appropriate times and typically alone (as will be described hereafter), to the session cup 110 from a supply or reservoir by a pump or flow control device or valve through fluid line, as described, for example in FIG. 1(a).

Add-on ingredients, or add-ons, in some circumstances, may also be added to the session cup 110 at appropriate times. In this regard, add-ons are provided directly to the session cup 110 from reservoirs 156 through fluid lines 154 via corresponding pumps 157. In this embodiment, there is no add-on cup or corresponding pump provided to the system.

Electrical connections between various components are not shown in FIG. 1(e) for ease of reference. As an example, the lines of FIG. 1(a) may be implemented. However, it should be appreciated that the actual connections or network may take a variety of different forms to suit the implementation or objectives of the system design. For example, hardwire connections may be implemented. Alternatively, or in combination, wireless connections may be established.

In operation, according to at least one form of the presently described embodiments, a liquid, high concentration DHA solution (Part A) of supply or reservoir 120 is used as the base tanning solution. A second solution with activating and optimizing components (Part B) from supply or reservoir 130 is used to dilute the high concentration DHA solution to provide the precise final concentration required for the desired tanning results. Generally, it should be appreciated that higher concentrations of DHA yield darker tans. The volume of Part B solution supplied from supply or reservoir 130 can be further modified to account for the presence of add-ons for fragrances, bronzers, accelerators or other solutions that provide skincare benefits or a faster tan. In this regard, by using less of the Part B solution to account for the liquid volume added by the optional add-ons, the concentration of DHA in the final solution mixture in the session cup 110 remains constant, yielding more consistent tanning results.

Dosing pumps, such as pumps 122, 132, and 157 provide a predetermined amount of solution based on the settings for the session and are described in greater detail in connection with FIG. 1(a).

With reference now to FIG. 1(f), another example system, system 100F, for spray tan customization using a multi-part solution is illustrated. As shown, the system 100F includes a controller 102 having at least one processor 104 and at least one memory 106, as described in connection with FIG. 1(a).

Also shown in FIG. 1(f) is a session cup or reservoir 110. The session cup 110 is configured and positioned to receive suitable amounts of solution and/or other fluids (such as moisturizers and add-ons) for a tanning session. According to the presently described embodiments, the tanning session is customized according to the preferences of the user or tanner. As such, the precise amounts of solution and/or other fluids will be controlled by the system (e.g., by way of the controller 102) and likely vary from session to session. In at least one form, the provision of the solutions to the session cup 110 has sufficient turbulence to sufficiently mix the solutions. That is, a mixing element or mixer is not provided. The solution is then dispensed appropriately through outlet 114. It should be appreciated that in at least one form, a solenoid switch 161 (or another suitable switch) is provided to allow water flow, at appropriate times, to the session cup 110. The water flow could be used to purge the session cup 110 and could be controlled in a variety of ways including, for example, by the control system or controller 102. It should also be appreciated that the solenoid switch (or another suitable switch) controlling water flow for purging, for example, the session coup could be implemented in other embodiments described herein (e.g., FIGS. 1(a)-1(e)).

As noted, solution and other fluids are provided to the session cup 110. For example, a first solution or Part A is provided to the session cup 110 from a supply or reservoir 120 by a pump or flow control device or valve 122 through fluid line 124. It will be appreciated that, in at least one form of the presently described embodiments, the Part A solution is a high concentration DHA solution. A second solution or Part B is provided to the session cup 110 from a supply or reservoir 130 by a pump or flow control device or valve 132 through fluid line 134. It will be appreciated that, in at least one form of the presently described embodiments, the Part B solution is a solution having activating and optimizing components.

The supply or reservoir 120 and the supply of reservoir 130 may take a variety of forms, as described in more detail in connection with FIG. 1(a). However, generally, the supply or reservoir 120 and the supply or reservoir 130 are configured to stage or store the first solution (or Part A solution) and the second solution (or Part B solution), respectively.

It will be further appreciated that, although not shown in FIG. 1(f), a moisturizer may be provided, in at least some forms, at appropriate times and typically alone (as will be described hereafter), to the session cup 110 from a supply or reservoir by a pump or flow control device or valve through fluid line, as described, for example in FIG. 1(a).

Add-on ingredients, or add-ons, in some circumstances, may also be added to the session cup 110 at appropriate times. In this regard, add-ons are provided directly to the session cup 110 from reservoirs 156 through fluid lines 154 via corresponding pumps 157. In this embodiment, there is no add-on cup or corresponding pump provided to the system.

Electrical connections between various components are not shown in FIG. 1(f) for ease of reference. As an example, the lines of FIG. 1(a) may be implemented. However, it should be appreciated that the actual connections or network may take a variety of different forms to suit the implementation or objectives of the system design. For example, hardwire connections may be implemented. Alternatively, or in combination, wireless connections may be established.

In operation, according to at least one form of the presently described embodiments, a liquid, high concentration DHA solution (Part A) of supply or reservoir 120 is used as the base tanning solution. A second solution with activating and optimizing components (Part B) from supply or reservoir 130 is used to dilute the high concentration DHA solution to provide the precise final concentration required for the desired tanning results. Generally, it should be appreciated that higher concentrations of DHA yield darker tans. The volume of Part B solution supplied from supply or reservoir 130 can be further modified to account for the presence of add-ons for fragrances, bronzers, accelerators or other solutions that provide skincare benefits or a faster tan. In this regard, by using less of the Part B solution to account for the liquid volume added by the optional add-ons, the concentration of DHA in the final solution mixture in the session cup 110 remains constant, yielding more consistent tanning results.

Dosing pumps, such as pumps 122, 132, and 157 provide a predetermined amount of solution based on the settings for the session and are described in greater detail in connection with FIG. 1(a). Check valves 159 (or other suitable valves) are provided in the lines 154 between the pumps 157 and the session cup 110. It should be appreciated that check valves (or other suitable elements) could be used on all lines of all embodiments described herein, if desired. However, in the embodiment of FIG. 1(f) (and, for example, FIG. 1(e)), the check valves 159 have a particular benefit where the physical orientation of the add-ons relative to the session cup 110 would allow gravity to cause the add-ons to drip into the session cup in an unwanted manner. The check valves (or other suitable elements) prevent the undesired dripping of add-ons into the session cup.

It should be appreciated that while all described embodiments (and variations) have advantages and benefits, the embodiment described in connection with FIG. 1(f) includes the benefit of efficiency in cost and process by eliminating the add-on cup to allow for a single mix session. This configuration also allows for ease of rinsing and/or purging because the rinsing and/or purging can be applied in one location, the session cup. As such, only a single rinse and/or purge is needed and/or desired in many applications.

It should be appreciated that, although mixers or mixing elements, such as a mixer or mixing element 112, are not shown in FIGS. 1(b)-1(f), mixers or mixing elements may be added to such systems. Such additional elements could be added as alternatives to the techniques/systems described or as supplemental or optional features.

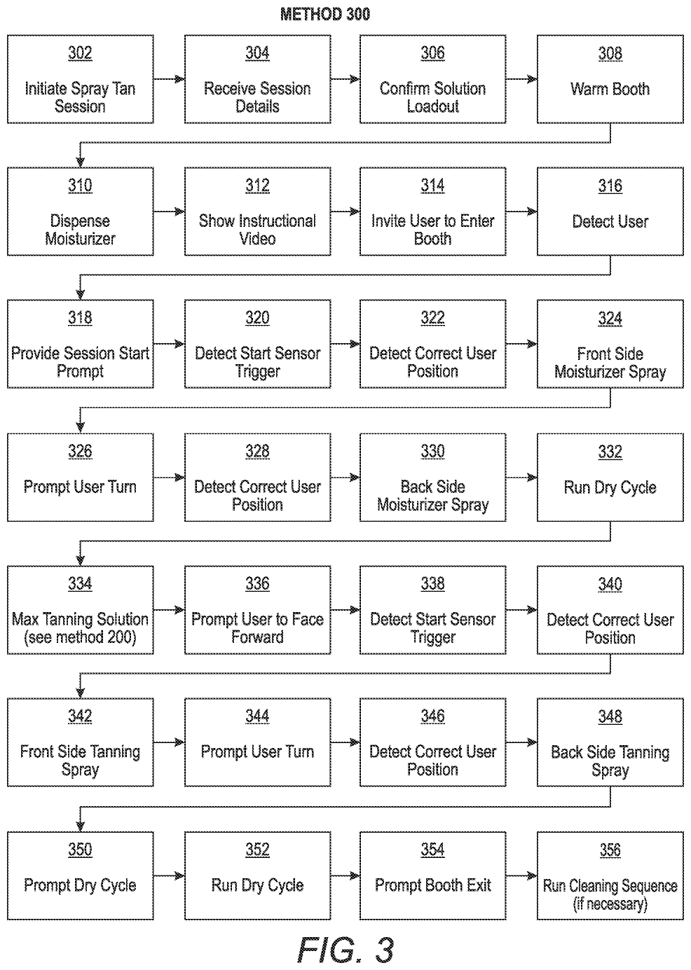

Now, with reference to FIG. 2, an example method 200, according to the presently described embodiments, for spray tan customization using a multi-part solution is illustrated. It should be appreciated that this method 200 may be implemented using systems described in connection with FIGS. 1(a)-1(f) or any other suitable system. It should also be recognized that the method 200 and other methods and techniques according to the presently described embodiments (including the method of FIG. 3) may be implemented using a variety of different configurations of hardware, software code or instructions (e.g., stored on appropriate non-volatile memory devices and/or non-transitory computer-readable medium and executed by suitable processors) and other functional components. For example, various hardware configurations and software routines may be used to trigger or cause the various components of the system to perform necessary functions to achieve the objectives of the presently described embodiments.

As shown, the method 200, a mix sequence, is initiated (at 202). The mix sequence is initiated by the controller 102 based on user or tanner input through an appropriate interface. Next, add-ons from the various supply elements that are selected by a user or tanner, are dispensed to the add-on cup 150 through supply lines 158 (at 204). This, too, is controlled by the controller based on the selections. It should be appreciated, however, that some embodiments, for example, the embodiments described in connection with FIG. 1(e) and FIG. 1(f), the add-ons will be dispensed directly from the reservoir to the session cup.

It should be appreciated that each add-on will typically have a volume of approximately 1 ml to 4 ml. The total volume of the add-on cup 150, in at least one form, will be approximately 20 ml. Accordingly, in some applications, the number and/or amounts of add-ons may have a practical or specified limit. Further, this portion of the mix sequence is optional inasmuch as it is driven by user/tanner selections. If no add-on selections are made, there is no need to dispense add-ons.

The first solution or Part A solution is dispensed from the supply or reservoir 120 to the session cup 110 by the pump or fluid control device or valve 122 through fluid line 124 (at 206). As noted above, in at least one form, this solution is high concentration DHA solution. The amount of Part A solution dispensed is controlled by the controller 102 based on selections of the user/tanner and any necessary calculations. Also, as noted above, the volume of the Part A solution may be modified according to the content of the add-on cup 150. Further, the controller activates the pump 122 to dispense the first solution or Part A solution into the session cup 110.

Likewise, the second solution or Part B solution is dispensed from the supply or reservoir 130 to the session cup 110 by the pump or fluid control device or valve 132 through fluid line 134 (at 208). The dispensing of the Part B solution may be accomplished concurrently with the dispensing of Part A solution or at a time different than the Part A solution. In at least one form, the timing of the dispensing of Part A and Part B solutions (and, possibly, add-on solutions as described,) may be a function of the mixing technique that is implemented. That is, for example, concurrent dispensing may be implemented where desired turbulence for mixing is achieved by the resultant concurrent flow of Part A and Part B (and other solutions, where applicable) into the session cup. As noted above, in at least one form, this solution comprises activating and optimizing components. The amount of Part B solution dispensed is controlled by the controller 102 based on selections of the user/tanner and any necessary calculations. Also, as noted above, the volume of the Part B solution may be modified according to the content of the add-on cup 150. Further, the controller activates the pump 132 to dispense the second solution or Part B solution into the session cup 110. It should be appreciated, however, that variations of this method may be implemented. For example, as with the embodiment described in connection with FIG. 1(c), the Part B solution may be indirectly routed to the session cup 110, that is, for example, routed through the add-on cup 150 before flowing to the session cup 110.

If add-ons were selected and the add-on cup 150 is present and has content, the contents of the add-on cup 150 are dispensed to the session cup 110 by the pump or fluid control device or valve 152 through fluid lines 154 (at 210). Further, the controller activates the pump 152 to dispense the add-ons into the session cup 110. Of course, if no add-on cup is provided to the system, there is no need to dispense the contents of the add-on cup.

Once the dispensing to the session cup 110 is complete, the session cup mixer or mixing element 112 is activated to mix the contents of the session cup 110 (at 212). In this regard, a variety of forms of agitation may be used to mix the solutions. For example, physical, vibrational or acoustic agitation may be used.

Of course, in at least some embodiments, such as those described in connection with FIGS. 1(b) through 1(f), a mixing element or mixer is not used. In these forms, the solutions are mixed by virtue of the turbulence generated during delivery of the solutions to the session cup 110. In this regard, in at least one form, the pumps from the various reservoirs will pump the solutions at a level so sufficient flow velocity or flow rate (e.g., greater than 1 ml/s such as approximately 3 ml/s) is achieved to mix the solutions as they are dispensed into the session cup 110. The mix sequence then ends (at 214), and the suitably mixed contents of the session cup 110 are ready to be dispensed in an appropriate manner according to the implementation.

In this regard, one commercial use of the system of the presently described embodiments is as part of an automated sunless spray tanning booth. Such booths typically utilize custom spray nozzles to supply a metered dose of the chemical DHA that is used to provide a UV-free tanning experience. Such a system could be designed with or adapted (or retrofitted) with a system according to the presently described embodiments.

With reference to FIG. 3, a method for 300 for obtaining a customized spray tan is illustrated. It will be understood that the method 300 is merely an example of an implementation of the presently described embodiments. Obviously, many variations are possible to achieve the presently described embodiments.

As shown, the spray tan session is initiated (at 302). Next, the system receives session details based on the user/tanner inputs and any necessary calculations (at 304). Solution loadout is confirmed (at 306). The booth is warmed (at 308) using appropriate techniques. Moisturizer is dispensed into the session cup 110 (at 310). An instructional video is then optionally show to the user/tanner (at 312). The user is invited to enter the booth (at 314) and the user is detected (at 316). Once the user is detected, a session start prompt is provided (at 318) and a start sensor trigger is detected (at 320). Once it is detected that the user is in a correct position (at 322), a front side moisturizer spray is completed (at 324). The user is then prompted to turn (at 326). Again, once is it detected that the user is in a correct position (at 328), a back side moisturizer spray is completed (at 330). A dry cycle is then run (at 332).

Next, as described in connection with FIGS. 1(a)-1(f) and 2, a mix sequence for the tanning solution is run (at 334). Once the mix sequence is complete, the user is prompted to face forward (at 336). A start sensor trigger is detected (at 338). If the user is detected in a correct position (at 340), front side spray tanning is completed (at 342). The user is then prompted to turn (at 344) and, if the user is detected in a correct position (at 346), back side spray tanning is completed (at 348). A dry cycle is prompted (at 350) and run (at 352). After the dry cycle completes, the user is prompted to exit the booth (at 354). A cleaning sequence is then run (if necessary) (at 356).

With reference back to FIGS. 1(a)-1(f) and 2, another commercial application of the presently described embodiments is as part of a hand-held spray gun system. In such an application, in at least one form, the system (e.g., systems 100A-100F) as described would mix spray solutions as described in connection with method 200 of FIG. 2 (with possible appropriate modifications). However, rather than be dispensed to a booth for application to the user/tanner, the content of the session cup 110 would be dispensed to a supply line of a hand-held spray gun that is typically utilized by trained technicians to apply bespoke tanning results to the customer.

As can be seen from the description above, the presently described embodiments provide many advantages over the current state of the art in the spray tan field. In this regard, according to the presently described embodiments, rather than requiring a provider of tanning services to stock multiple individual tan level solutions, a single Part A and a single Part B reservoir are sufficient to perform a whole range of tans. This makes stock and inventory easier to manage while also ensuring high DHA solution throughput. This is beneficial since DHA in solution has a fixed shelf life before it becomes less effective at providing a tanning result.

At least some other advantages of the presently described embodiments include:
Greater customization
Finer control of tan darkness
More precise tanning results
More consistent tanning results
Higher solution throughput (for a fixed number of tans)
Less complicated inventory tracking
Automated inclusion of optional add-ons

The exemplary embodiment has been described with reference to the preferred embodiments. Obviously, modifications and alterations will occur to others upon reading and understanding the preceding detailed description. It is intended that the exemplary embodiment be construed as including all such modifications and alterations insofar as they come within the scope of the appended claims or the equivalents thereof.

1. A system for spray tan customization using a multi-part solution, the system comprising:
a first reservoir configured to stage a first solution comprising dihydroxyacetone (DHA), the first reservoir being provided with a first pump;
a second reservoir configured to stage a second solution comprising activating components including amino acids, the second reservoir being provided with a second pump;
a third reservoir configured to receive the first and second solution; and,
a controller including at least one processor and at least one memory, wherein the at least one memory stores code or instructions that can be executed by the at least one processor to initiate a mix sequence for the first solution staged in the first reservoir and the second solution staged in the second reservoir based on user preferences for a spray tan session, activate the first pump to dispense the first solution from the first reservoir to a third reservoir based on the user preferences, and activate the second pump to dispense the second solution from the second reservoir to the third reservoir based on the user preferences, wherein the first solution and the second solution are mixed in the third reservoir.

2. The system as set forth in claim 1 further comprising a mixing element configured to mix contents of the third reservoir.

3. The system as set forth in claim 2 wherein the controller is further configured to activate the mixer to mix the first solution and the second solution in the third reservoir.

4. The system as set forth in claim 1 further comprising a fourth reservoir to collect additional ingredients, the fourth reservoir being provided with a third pump.

5. The system as set forth in claim 4 further comprising a check valve disposed between the third pump and the third reservoir.

6. The system as set forth in claim 1 further comprising a solenoid switch connected to the third reservoir, the solenoid switch being configured to trigger water flow into the third reservoir.

7. The system as set forth in claim 4 wherein the second pump is activated to dispense the second solution to the third reservoir through the fourth reservoir.

8. The system as set forth in claim 4 wherein the third pump is activated by the controller to dispense contents of the fourth reservoir to the third reservoir based on the user preferences.

9. The system as set forth in claim 1 further comprising a fifth reservoir including moisturizer, the fifth reservoir being provided with a fourth pump.

10. The system as set forth in claim 9 wherein the fourth pump is selectively activated by the controller to dispense contents of the fifth reservoir to the third reservoir.

11. The system as set forth in claim 1 wherein mixed contents of the third reservoir are provided to a spray tan booth.

12. The system as set forth in claim 1 wherein mixed contents of the third reservoir are provided to a spray gun.

13. A method for spray tan customization using multi-part solution, the method comprising:
initiating a mix sequence for a first solution comprising dihydroxyacetone (DHA) staged in a first reservoir and a second solution comprising activating components comprising amino acids staged in a second reservoir based on user preferences for a spray tan session;
dispensing the first solution from the first reservoir to a third reservoir based on the user preferences;
dispensing the second solution from the second reservoir to the third reservoir based on the user preferences; and,
mixing the first solution and the second solution in the third reservoir.

14. The method as set forth in claim 13 wherein the mixing comprises activating a mixer to mix the first solution and the second solution in the third reservoir.

15. The method as set forth in claim 13 further comprising collecting additional ingredients in a fourth reservoir.

16. The method as set forth in claim 15, wherein the dispensing the second solution from the second reservoir to the third reservoir comprises dispensing the second solution through the fourth reservoir to the third reservoir.

17. The method as set forth in claim 15 further comprising dispensing contents from the fourth reservoir to the third reservoir based on the user preferences.

18. The method as set forth in claim 13 further comprising dispensing moisturizer.

19. The method as set forth in claim 13 further comprising providing mixed contents of the third reservoir to a spray tan booth.

20. The method as set forth in claim 13 further comprising providing contents of the third reservoir to a spray gun.
